(19) United States
(12) Patent Application Publication
(10) Pub. No.: US 2014/0004087 A1
(43) Pub. Date: Jan. 2, 2014

(54) ENDOMETRIAL REGENERATIVE CELLS FOR TREATMENT OF TRAUMATIC BRAIN INJURY

(71) Applicant: Medistem, Inc., (US)

(72) Inventors: Thomas Ichim, San Diego, CA (US); Vladimir Bogin, Portland, OR (US)

(73) Assignee: Medistem, Inc., San Diego, CA (US)

(21) Appl. No.: 13/854,056

(22) Filed: Mar. 30, 2013

Related U.S. Application Data
(60) Provisional application No. 61/618,974, filed on Apr. 2, 2012.

(51) Int. Cl. C12N 5/071

(52) U.S. Cl. CPC C12N 5/0682
USPC 424/93.7

(57) ABSTRACT

The use of endometrial regenerative cells (ERC) and other endometrial originating cells for the treatment of traumatic brain injury is disclosed. In one embodiment a patient is administered a population of CD90 positive, CD105 positive, allogeneic regenerative cells subsequent to a brain injury. Cell concentration, frequency of administration, and route of administration may be determined based on extent of injury, inflammatory response and endogenous stem cell mobilization. In one embodiment, a patient suffering from traumatic brain injury is administered a dose of 100 million Endometrial Regenerative Cells intravenously at a rate of 1 million cells per minute in a volume of 100 ml of saline.

### CROSS-REFERENCE TO RELATED APPLICATIONS

This application claims priority to Provisional Application Ser. No. 61/618,974, filed Apr. 2, 2012, and entitled “Endometrial Regenerative Cells for Treatment of Traumatic Brain Injury” which is hereby expressly incorporated by reference in its entirety.

### FIELD OF THE INVENTION

The invention pertains to the field of brain injuries, more particularly, the invention relates to the field of treating brain injuries through administration of a cellular population possessing a high angiogenic activity. More particularly, the invention pertains to treatment of brain injuries through administration of Endometrial Regenerative Cells (ERC).

### BACKGROUND

Injury to central nervous system (CNS) induced by acute insults including trauma, hypoxia and ischemia (caused by stroke or blunt force trauma) can affect both grey and white matter dependent on nature and severity. Injury to CNS involves neuro-inflammation. For example, leukocyte infiltration in the CNS after trauma or inflammation is triggered in part by up-regulation of the MCP-1 chemokine in astrocytes. Injury is often self-perpetuated by inflammatory mediators released that in turn activate toll like receptors [1]. Trauma is an injury or damage of the nerve. It may be spinal cord trauma, which is damage to the spinal cord that affects all nervous functions that are controlled at and below the level of the injury, including muscle control and sensation, or brain trauma, such as trauma caused by closed head injury. Cerebral hypoxia is a lack of oxygen specifically to the cerebral hemispheres, and more typically the term is used to refer to a lack of oxygen to the entire brain. Depending on the severity of the hypoxia, symptoms may range from confusion to irreversible brain damage, coma and death. Stroke is usually caused by reduced blood flow (ischemia) of the brain. It is also called cerebrovascular disease or accident. It is a group of brain disorders involving loss of brain functions that occurs when the blood supply to any part of the brain is interrupted.

The brain requires about 20% of the circulation of blood in the body. The primary blood supply to the brain is through 2 arteries in the neck (the carotid arteries), which then branch off within the brain to multiple arteries that each supply a specific area of the brain. Even a temporary interruption to the blood flow can cause decreases in brain function (neurological deficit). The symptoms vary with the area of the brain affected and commonly include such problems as changes in vision (occipital lobe), speech changes (Broca' s Area), decreased movement or sensation in a part of the body (cerebellum), or changes in the level of consciousness (temporal lobe). If the blood flow is decreased for longer than a few seconds, brain cells in the area are destroyed (infarcted) causing permanent damage to that area of the brain or even death.

Stroke affects about 4 out of 1,000 people. It is the 3rd leading cause of death in most developed countries, including the U.S. The incidence of stroke rises dramatically with age, with the risk doubling with each decade after age 35. About 5% of people over age 65 have had at least one stroke. The disorder occurs in men more often than women. Causes of ischemic strokes are blood clots that form in the brain (thrombus) and blood clots or pieces of atherosclerotic plaque or other material that travel to the brain from another location (emboli). Bleeding (hemorrhage) within the brain may cause symptoms that mimic stroke. Strokes secondary to atherosclerosis (cerebral thrombosis) and strokes caused by embolism (moving blood clot) are the most common strokes. Traumatic nerve injury may concern both the CNS or the PNS. Traumatic brain injury, also simply called head injury or closed head injury, refers to an injury where there is damage to the brain because of an external blow to the head. It mostly happens during car or bicycle accidents, but may also occur as the result of near drowning, heart attack, stroke and infections. In military combat TBI is of particular relevance according to a recent report, between 2000 to 2010, there have been 178,876 cases of TBI (The Defense and Veterans Brain Injury Center, http://www.dvbic.org/TBI-Numbers.aspx). In the civilian population, according to the CDC there are between 1.5 and 2 million Americans that suffer TBI every year (“Traumatic Brain Injury,” Center for Disease Control and Prevention, National Center for Injury Prevention and Control, 2003, Vol. 2003). In the same reported, it is stated that TBI is responsible for 50,000 deaths and 100,000 hospitalizations annually. Additionally, over 80,000 individuals are disabled annually, approximately 17,000 of whom require specialized care for life. This type of traumatic brain injury would usually result due to the lack of oxygen or blood supply to the brain, and therefore can be referred to as an “anoxic injury”. Brain injury or closed head injury occurs when there is a blow to the head as in a motor vehicle accident or a fall. There may be a period of unconsciousness immediately following the trauma, which may last minutes, weeks or months. Primary brain damage occurs at the time of injury, mainly at the sites of impact, in particular when a skull fraction is present.

Large contusions may be associated with an intracerebral haemorrhage, or accompanied by cortical lacerations. Diffuse axonal injuries occur as a result of shearing and tensile strains of neuronal processes produced by rotational movements of the brain within the skull. There may be small heamorrhagic lesions or diffuse damage to axons, which can only be detected microscopically. Secondary brain damage occurs as a result of complications developing after the moment of injury. They include intracranial hemorrhage, traumatic damage to extracerebral arteries, intracranial herniation, hypoxic brain damage or meningitis.

Intracerebral hemorrhage (ICH) is a devastating type of stroke with high mortality and morbidity rate, effects 40 000 people in the United States annually [2]. Additionally, as stated above, ICH may be the result of blunt force trauma [3]. Despite of significant progress in diagnostics of this disease and on-going research on new therapy strategies there is no cure significantly decreasing mortality and improving life quality of survivals. Only fifty percent of patients will survive ICH, and these individuals will be afflicted with significant brain atrophy and lifelong neurological deficits [4]. Primary bleeding initiates several pathophysiological pathways leading to the secondary brain injury. This pathways include activation of microglia [5], production of pro-inflammatory cytokines [6] and reactive oxygen species [7] and infiltration by systemic immune cells [8]. Inflammation and ROS production are known events leading to disruption of blood-brain barrier, development of brain edema, the most live-threatened event after ICH. The long-term effect of factors mentioned above is a brain atrophy and long-lasting neurological deficit [9, 10].

Recent publications demonstrated that the brain after ICH is capable to self-repair [11]. It was also demonstrated that some endogenous and exogenous factors can stimulate post-injury neurogenesis and angiogenesis and promote brain recovery after ICH resulting in significant improvement of neurological outcome [12, 13].

Intracerebral hemorrhage is one of the most deadly kind of stoke accounting for approximately 5 to 15% of all types of stroke. ICH is more than twice more common as subarachnoid hemorrhage (SAH) and results in more disability and death than SAH or ischemic stroke. Despite of significant progress in diagnostics of this disease and on-going research on new therapy strategies there is no cure significantly decreasing mortality and improving life quality of survivals. The continuum of injury, whether by blunt force trauma, or cerebrovascular accident, all culminates into ischemic damage. Unfortunately, to date, with exception of thrombolytics, all clinical trials in stroke have failed. Agents tested included including antioxidants, calcium channel blockers, glutamate receptor blockers, and neurotrophic factors in over 1000 clinical trials [14]. Accordingly, there is a major need in the field for new approaches towards brain insults.

### SUMMARY OF THE INVENTION

Certain embodiments are directed to a cell composition useful for the treatment of traumatic brain injury comprising a cell originating from the endometrium, said cell expresses a marker selected from CD29, CD41a, CD44, CD90, and CD105, and having an ability to proliferate at a rate of 0.5-1.5 doublings per 24 hours in a growth medium.

The cell composition can further express a marker selected from NeuN, CD9, CD62, CD59, Actin, GFAP, NSE, Nestin, CD73, SSEA-4, hTERT, Oct-4, and tubulin. The cell composition can further express a marker selected from hTERT and Oct-4, but does not express a STRO-1 marker, and has an ability to undergo cell division in less than 24 hours in a growth medium. The cell composition can further express a STRO-1 marker, and has an ability to proliferate at a rate of 0.5-0.9 doublings per 24 hours in a growth medium. The cell composition can further produce matrix metalloprotease 3 (MMP3), matrix metalloprotease 10 (MMP10), GM-CSF, PDGF-BB or angiogenic factor ANG-2. Further embodiments include endometrial regenerative cells expressing substantially higher amounts of the following genes as compared to bone marrow derived mesenchymal stem cells: somatostatin receptor 1, forkhead box L2, FAM105A, synaptopodin 2-like, anoctamin 4, spondin 2, CARD16, VAT1L, indolethylamine N-methyltransferase, deiodinase, iodothyronine, type II (DIO2), aldehyde dehydrogenase 1 family, member A1, 5-hydroxytryptamine (serotonin) receptor 2B, caspase recruitment domain family, member 17, Rho GTPase activating protein 20, zinc finger and BTB domain containing 46 (ZBTB46), synaptopodin 2-like (SYNPO2L), transcript variant 1, mRNA, chromosome 13 open reading frame 15 (C13orf15), mRNA, homeobox D11 (HOXD11), mRNA, oxidized low density lipoprotein (lectin-like) receptor 1 (OLR1), transcript variant 2, mRNA, homeobox D10 (HOXD10), mRNA, Pregnancy specific beta-1-glycoprotein 4, matrix metallopeptidase 3 (stromelysin 1, progelatinase) (MMP3), mRNA, actin filament associated protein 1-like 2 (AFAP1L2), transcript variant 2, mRNA, SH3-domain GRB2-like (endophilin) interacting protein 1 (SGIP1), mRNA, solute carrier family 7 (cationic amino acid transporter, y+ system), member 2 (SLC7A2), transcript variant 1, mRNA, pregnancy specific beta-1-glycoprotein 8 (PSG8), transcript variant 3, mRNA, Wilms tumor 1 (WT1), transcript variant F, mRNA, platelet-derived growth factor beta polypeptide (PDGFB), transcript variant 1, mRNA, forkhead box F1 (FOXF1), mRNA, mannan-binding lectin serine peptidase 1 (C4/C2 activating component of Ra-reactive factor) (MASP1), transcript variant 1, mRNA, deiodinase, iodothyronine, type II (DIO2), transcript variant 4, mRNA, G protein-coupled receptor 126 (GPR126), transcript variant b1, mRNA, stimulated by retinoic acid gene 6 homolog (mouse) (STRA6), transcript variant 6, mRNA, hydroxysteroid (11-beta) dehydrogenase 1 (HSD11B1), transcript variant 2, mRNA, RAS guanyl releasing protein 2 (calcium and DAG-regulated) (RASGRP2), transcript variant 4, mRNA, membrane bound O-acyltransferase domain containing 1 (MBOAT1), mRNA, LON peptidase N-terminal domain and ring finger 2 (LONRF2), mRNA, interferon-induced protein with tetratricopeptide repeats 2 (IFIT2), mRNA, cathepsin C (CTSC), transcript variant 3, mRNA, caspase 1, apoptosis-related cysteine peptidase (interleukin 1, beta, convertase) (CASP1), transcript variant beta, mRNA, integrin, alpha 4 (antigen CD49D, alpha 4 subunit of VLA-4 receptor) (ITGA4), mRNA, integrin, alpha 6 (ITGA6), transcript variant 1, mRNA, contactin 3 (plasmacytoma associated) (CNTN3), mRNA, synaptophysin-like 2 (SYPL2), mRNA, Rho GTPase activating protein 25 (ARHGAP25), transcript variant 4, mRNA, potassium voltage-gated channel, Shal-related subfamily, member 2 (KCND2), mRNA, S100 calcium binding protein A4 (S100A4), transcript variant 1, mRNA, chemokine (C-C motif) receptor-like 2 (CCRL2), transcript variant 2, mRNA, integrin, alpha 6 (ITGA6), transcript variant 2, mRNA, guanylate binding protein 4 (GBP4), mRNA, multiple C2 domains, transmembrane 1 (MCTP1), transcript variant S, mRNA, interferon-induced protein with tetratricopeptide repeats 2 (IFIT2), mRNA, Clq and tumor necrosis factor related protein 9 (C1QTNF9), mRNA, progesterone receptor (PGR), transcript variant 2, mRNA, hypothetical LOC646113 (FLJ43390), non-coding RNA. zinc finger and BTB domain containing 46 (ZBTB46), mRNA, interferon-induced protein with tetratricopeptide repeats 3 (IFIT3), transcript variant 1, mRNA, microphthalmia-associated transcription factor (MITF), transcript variant 2, mRNA, interleukin 24 (IL24), transcript variant 4, mRNA, neuronal pentraxin I (NPTX1), mRNA, integrin, alpha 4 (antigen CD49D, alpha 4 subunit of VLA-4 receptor) (ITGA4), mRNA, WT1 antisense RNA (non-protein coding) (WT1-AS), non-coding RNA, microphthalmia-associated transcription factor (MITF), transcript variant 2, mRNA, sterile alpha motif domain containing 12 (SAMD12), transcript variant 1, mRNA, chromosome 7 open reading frame 58 (C7orf58), transcript variant 1, mRNA, phospholipase B domain containing 1 (PLBD1), mRNA, podocalyxin-like (PODXL), transcript variant 2, mRNA, interleukin 8 (IL8), mRNA, chromosome 7 open reading frame 58 (C7orf58), transcript variant 2, mRNA, protein kinase, cAMP-dependent, regulatory, type II, beta (PRKAR2B), mRNA, chromosome 8 open reading frame 4 (C8orf4), mRNA, stimulated by retinoic acid gene 6 homolog (mouse) (STRA6), transcript variant 5, mRNA, progesterone receptor (PGR), transcript variant 2, mRNA, angiopoietin 2 (ANGPT2), transcript variant 2, mRNA, synaptophysin-like 2 (SYPL2), mRNA, retinoic acid receptor responder (tazarotene induced) 2 (RARRES2), mRNA, claudin 1 (CLDN1), mRNA, sal-like 1 (Drosophila) (SALL1), transcript variant 1, mRNA, calcium channel, voltage-dependent, T type, alpha 1H subunit (CACNA1H), transcript variant 2, mRNA, armadillo repeat containing 4 (ARMC4), mRNA, phosphatidylinositol-specific phospholipase C, X domain containing 3 (PLCXD3), mRNA, G protein-coupled receptor, family C, group 5, member A (GPRC5A), mRNA, GATA binding protein 2 (GATA2), transcript variant 1, mRNA, actin filament associated protein 1-like 1 (AFAP1L1), transcript variant 1, mRNA, ST6 (alpha-N-acetyl-neuraminyl-2,3-beta-galactosyl-1,3)-N-acetylgalactosaminide alpha-2,6-sialyltransferase 5 (ST6GALNAC5), mRNA, H19, imprinted maternally expressed transcript (non-protein coding) (H19), non-coding RNA, sphingosine-1-phosphate receptor 3 (S1PR3), mRNA, protein kinase, cAMP-dependent, regulatory, type II, beta (PRKAR2B), mRNA, interleukin 7 receptor (IL7R), mRNA, calcium/calmodulin-dependent protein kinase IG (CAMK1G), mRNA, homeobox A11 (HOXA11), mRNA, renin (REN), mRNA, transcription factor AP-2 gamma (activating enhancer binding protein 2 gamma) (TFAP2C), mRNA, cathepsin C (CTSC), transcript variant 2, mRNA, STAM binding protein-like 1 (STAMBPL1), mRNA, Hypothetical protein LOC254057, adrenomedullin (ADM), mRNA, tenascin XB (TNXB), transcript variant XB-S, mRNA, KH domain containing, RNA binding, signal transduction associated 3 (KHDRBS3), mRNA, solute carrier family 35, member F3 (SLC35F3), mRNA, collagen, type IV, alpha 6 (COL4A6), transcript variant B, mRNA, protocadherin 7 (PCDH7), transcript variant a, mRNA, chromosome 10 open reading frame 58 (C10orf58), transcript variant 1, mRNA, fibroblast growth factor 9 (glia-activating factor) (FGF9), mRNA, sushi domain containing 3 (SUSD3), mRNA, melanoma associated antigen (mutated) 1-like 1 (MUM1L1), transcript variant 2, mRNA, keratin 18 pseudogene (F1140504), non-coding RNA, C-type lectin domain family 14, member A (CLEC14A), mRNA, 1-acylglycerol-3-phosphate O-acyltransferase 9 (AGPAT9), mRNA, phosphodiesterase 8B (PDE8B), transcript variant 3, mRNA, ArfGAP with dual PH domains 2 (ADAP2), mRNA, Keratin 18, family with sequence similarity 65, member C (FAM65C), mRNA, androgen receptor (AR), transcript variant 1, mRNA, phosphodiesterase 9A (PDE9A), transcript variant 2, mRNA, Intercellular adhesion molecule 1, monoglyceride lipase (MGLL), transcript variant 2, mRNA, HOXA11 antisense RNA 1 (non-protein coding) (HOXA11-AS1), antisense RNA, receptor (chemosensory) transporter protein 4 (RTP4), mRNA, reticulon 4 receptor (RTN4R), mRNA, Keratin pseudogene, annexin A3 (ANXA3), mRNA, RAS guanyl releasing protein 2 (calcium and DAG-regulated) (RASGRP2), transcript variant 3, mRNA, serum deprivation response (SDPR), mRNA, collagen, type XIII, alpha 1 (COL13A1), transcript variant 21, mRNA, v-ets erythroblastosis virus E26 oncogene homolog 2 (avian) (ETS2), mRNA, ADAM metallopeptidase domain 8 (ADAMS), transcript variant 3, mRNA, prostate collagen triple helix (PCOTH), transcript variant 1, mRNA, family with sequence similarity 124A (FAM124A), transcript variant 1, mRNA, interferon, alpha-inducible protein 27 (IF127), transcript variant 2, mRNA, hypothetical protein MGC16121 (MGC16121), non-coding RNA, egf-like module containing, mucin-like, hormone receptor-like 2 (EMR2), transcript variant 4, mRNA, RAS guanyl releasing protein 2 (calcium and DAG-regulated) (RASGRP2), transcript variant 3, mRNA, KH domain containing, RNA binding, signal transduction associated 3 (KHDRBS3), mRNA, chromosome 9 open reading frame 47 (C9orf47), transcript variant 1, mRNA, family with sequence similarity 162, member B (FAM162B), mRNA, sodium channel, voltage-gated, type IX, alpha subunit (SCN9A), mRNA, glutaredoxin (thioltransferase) (GLRX), transcript variant 2, mRNA, four jointed box 1 (Drosophila) (FJX1), mRNA, chromosome 10 open reading frame 58 (C10orf58), transcript variant 1, mRNA, archaelysin family metallopeptidase 1 (AMZ1), mRNA, laminin, alpha 5 (LAMAS), mRNA, myosin XVI (MYO16), transcript variant 2, mRNA, SH3-domain GRB2-like (endophilin) interacting protein 1 (SGIP1), mRNA, nuclear receptor subfamily 2, group F, member 1 (NR2F1), mRNA, GULP, engulfment adaptor PTB domain containing 1 (GULP1), mRNA, actin binding LIM protein family, member 3 (ABLIM3), mRNA, transmembrane protein 154 (TMEM154), mRNA, germ cell associated 1 (GSG1), transcript variant 1, mRNA, suppressor of cytokine signaling 2 (SOCS2), mRNA, GULP, engulfment adaptor PTB domain containing 1 (GULP1), mRNA, integrin, alpha 4 (antigen CD49D, alpha 4 subunit of VLA-4 receptor) (ITGA4), mRNA, DENN/MADD domain containing 2A (DENND2A), mRNA, interferon-induced protein with tetratricopeptide repeats 1 (IFIT1), transcript variant 2, mRNA, C-type lectin domain family 2, member B (CLEC2B), mRNA, hairy and enhancer of split 5 (Drosophila) (HESS), mRNA, chromosome 7 open reading frame 58 (C7orf58), transcript variant 2, mRNA, Keratin 18, cathepsin C (CTSC), transcript variant 1, mRNA, G protein-coupled receptor 183 (GPR183), mRNA, Mitogen-activated protein kinase kinase kinase 8 caspase recruitment domain family, member 9 (CARD9), transcript variant 2, mRNA, nidogen 1 (NID1), mRNA, adenosine monophosphate deaminase 3 (AMPD3), transcript variant 3, mRNA, opioid growth factor receptor-like 1 (OGFRL1), mRNA, interferon induced transmembrane protein 1 (9-27) (IFITM1), mRNA, interleukin 2 receptor, beta (IL2RB), mRNA, tripartite motif containing 14 (TRIM14), transcript variant 1, mRNA, acyl-CoA synthetase short-chain family member 1 (ACSS1), nuclear gene encoding mitochondrial protein, mRNA, RNA binding motif protein 24 (RBM24), transcript variant 3, mRNA, stathmin-like 2 (STMN2), transcript variant 1, mRNA, GULP, engulfment adaptor PTB domain containing 1 (GULP1), mRNA, secreted and transmembrane 1 (SECTM1), mRNA, betaine--homocysteine S-methyltransferase 2 (BHMT2), transcript variant 1, mRNA, lymphocyte-activation gene 3 (LAG3), mRNA, transmembrane protein 51 (TMEM51), transcript variant 1, mRNA, guanine nucleotide binding protein (G protein), gamma 11 (GNG11), mRNA, CD163 molecule-like 1 (CD163L1), mRNA, major histocompatibility complex, class I, F (HLA-F), transcript variant 2, mRNA, endoplasmic reticulum metallopeptidase 1 (ERMP1), mRNA, cytochrome b5 reductase 2 (CYB5R2), mRNA, mitogen-activated protein kinase kinase kinase 5 (MAP3K5), mRNA, interleukin 20 (IL20), mRNA, T-box 3 (TBX3), transcript variant 1, mRNA, adrenergic, alpha-1D-, receptor (ADRA1D), mRNA, leucine rich repeat containing 8 family, member C (LRRC8C), mRNA, folate receptor 3 (gamma) (FOLR3), mRNA, tumor necrosis factor receptor superfamily, member 21 (TNFRSF21), mRNA, actin binding LIM protein family, member 3 (ABLIM3), mRNA, CD44 molecule (Indian blood group) (CD44), transcript variant 7, mRNA, phosphatidylinositol-3,4,5-trisphosphate-dependent Rac exchange factor 1 (PREX1), mRNA, tripartite motif containing 14 (TRIM14), transcript variant 1, mRNA, aldehyde dehydrogenase 1 family, member A1 (ALDH1A1), mRNA, adaptor-related protein complex 1, mu 2 subunit (AP1M2), mRNA, integrin, alpha 1 (ITGA1), mRNA, mitogen-activated protein kinase kinase kinase 5 (MAP3K5), mRNA, serpin peptidase inhibitor, clade B (ovalbumin), member 2 (SERPINB2), transcript variant 2, mRNA, scavenger receptor class B, member 1 (SCARB1), transcript variant 1, mRNA, homeobox D9 (HOXD9), mRNA, G protein-coupled receptor, family C, group 5, member B (GPRC5B), mRNA, Rho guanine nucleotide exchange factor (GEF) 16 (ARHGEF16), mRNA, adrenergic, alpha-2A-, receptor (ADRA2A), mRNA, intercellular adhesion molecule 4 (Landsteiner-Wiener blood group) (ICAM4), transcript variant 2, mRNA, guanine nucleotide binding protein (G protein), gamma 4 (GNG4), transcript variant 1, mRNA, BCL2-like 10 (apoptosis facilitator) (BCL2L10), mRNA, monoglyceride lipase (MGLL), transcript variant 1, mRNA, Furry homolog (Drosophila) phorbol-12-myristate-13-acetate-induced protein 1 (PMAIP1), mRNA, transmembrane and tetratricopeptide repeat containing 1 (TMTC1), transcript variant 1, mRNA, collagen, type IV, alpha 5 (COL4A5), transcript variant 1, mRNA, disrupted in renal carcinoma 3 (DIRC3), non-coding RNA, Rho GDP dissociation inhibitor (GDI) beta (ARHGDIB), mRNA, oxytocin receptor (OXTR), mRNA, tumor necrosis factor receptor superfamily, member 6b, decoy (TNFRSF6B), mRNA, cytochrome b5 reductase 2 (CYB5R2), mRNA, peptidase M20 domain containing 2 (PM20D2), mRNA, shroom family member 3 (SHROOM3), mRNA, family with sequence similarity 46, member C (FAM46C), mRNA, nuclear factor of kappa light polypeptide gene enhancer in B-cells inhibitor, epsilon (NFKBIE), mRNA, tumor necrosis factor receptor superfamily, member 6b, decoy (TNFRSF6B), mRNA.

Further embodiments include cell compositions, wherein said endometrial regenerative cell is extracted from the endometrium by means of an endometrial biopsy. The endometrial regenerative cell can be extracted from the menstrual blood as well. Additionally, the endometrial regenerative cell can be extracted from the menstrual blood by means of density centrifugation. The endometrial regenerative cell can be extracted from the menstrual blood by means of culture expansion. Additionally, the traumatic brain injury can be treated by intravenous administration of endometrial regenerative cells. The traumatic brain injury can be treated by intrathecal administration of said endometrial regenerative cells. The traumatic brain injury can be treated by intranasal administration of said endometrial regenerative cells. The traumatic brain injury can be treated by stereotactic administration of said endometrial regenerative cells. The traumatic brain injury can also be treated by stereotactic administration of said endometrial regenerative cells. The traumatic brain injury can be treated by administration of said endometrial regenerative cells within 1-48 hours, or 1-24 hours after said injury. The traumatic brain injury can be treated by administration of endometrial regenerative cells in a chronic manner, by repeat administration, by bolus administration, or by continuous administration after said injury. The traumatic brain injury can be treated by administration of approximately 100 million endometrial regenerative cells intravenously.

Additional embodiments are directed to methods of treating a neurological injury comprising administering into a patient an effective amount of endometrial regenerative cells.

### BRIEF DESCRIPTION OF THE DRAWINGS

FIG. 1 is a photograph of a mouse brain. Bleeding caused by the collagenase injection. Coronal section of the mouse brain along the needle track one hour after bacterial collagenase injection showed hematoma in the ipsilateral basal ganglia.

FIG. 2 is a bar graph showing the results of brain water content evaluation. 72 hours after ICH induction, ICH vehicle-treated animals (black bar, N=6), demonstrated elevated brain water content compared to sham-operated animals (white bar, N=4). ERC treatment demonstrated a strong tendency to decrease the ICH-induced elevation of brain water content (blue bar, N=4)

FIG. 3 is a graph showing the results of neurological evaluation: Modified Garcia Test. Significant neurological deficits were observed in all animals with collagenase-induced ICH compared to sham operated animals (N=4). ERC treatment, started one hour after ICH (N=4) when compared to vehicle treatment (N=6) tends to ameliorate neurological deficits at all tested time points

FIG. 4 is a graph showing the results of neurological evaluation: Limb placing test. Significant neurological deficits were observed in all animals with collagenase-induced ICH compared to sham operated animals (N=4). ERC treatment, started one hour after ICH (N=4) when compared to vehicle treatment (N=6) tends to ameliorate neurological deficits at all tested time points

FIG. 5 is a graph showing the results of neurological evaluation: Wire hanging test. Significant neurological deficits were observed in all animals with collagenase-induced ICH compared to sham operated animals (N=4). ERC treatment, started one hour after ICH (N=4) when compared to vehicle treatment (N=6) tends to ameliorate neurological deficits at all tested time points

### DETAILED DESCRIPTION OF THE ILLUSTRATED EMBODIMENTS

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Methods of treating a subject with a traumatic central nervous system injury, more particularly, a traumatic brain injury, are provided. The methods comprise treatment of a traumatic brain injury in a human subject by administering to the subject in need thereof a therapeutically effective concentration of Endometrial Regenerative Cells. In one embodiment, the invention teaches the treatment of traumatic brain injury through the administration of use of an endometrially-derived cells that have been generated to express a unique gene transcription pattern. Specifically, endometrial regenerative cells (ERC) may be purified, isolated, expanded, or extracted using protocols already disclosed that describe ERC [15], or cells we claim as ERC, specifically Endometrial or Menstrual Blood derived CD90, CD105 positive cells [16-19], or protocols described here, and purified for higher activity based on markers disclosed in the current invention such as CD73, somatostatin receptor 1, spondin 2, aldehyde dehydrogenase, or anoctamin 4. The selection of these cells may be performed using means known in the art such as flow cytometry sorting, magnetic activated cell sorting, panning, or other affinity-based purification means. A wealth of literature exists describing to one of skill in the art methods of isolating endometrial stem cells. Cui et al. [20], who used menstrual blood as a starting source for cell culture. They grew out adherent cells from a mononuclear preparation of screened menstrual blood that possessed a mesenchymal stem cell (MSC)-like morphology. The cells were positive for CD13, CD29, CD44, CD54, CD55, CD59, CD73, CD90, CD105 and STRO-1, and lacked CD14, CD34, CD45, CD133, and c-kit. The investigators demonstrated in vitro myocyte generation and myotubule formation, as well as in vivo ability to differentiate into dystrophin expressing muscle tissue in MDX-SCID mice.

The first demonstration of pluripotent stem cells derived from the endometrium occurred almost simultaneously by two independent groups. Meng et al [15], used the process of cloning rapidly proliferating adherence cells derived from menstrual blood and generated a homogenous cell population expressing CD9, CD29, CD41a, CD44, CD59, CD73, CD90, and CD105 and lacking CD14, CD34, CD45 and STRO-1 expression. In contrast to Cui et al, the authors demonstrated the cells had substantially faster replicative potential as compared to bone marrow MSC, a unique cytokine and MMP profile, as well as ability to differentiate into cardiomyocytic, respiratory epithelial, neurocytic, myocytic, endothelial, pancreatic, hepatic, adipocytic, and osteogenic lineages. Interestingly, the cells identified expressed telomerase and OCT-4 but lacked expression of NANOG-1. Given the pluripotent nature of these cells, the authors named them “Endometrial Regenerative Cells” (ERC). Shortly after, Patel et al [21] reported a population of cells isolated using c-kit selection of menstrual blood mononuclear cells. The cells had a similar phenotype, proliferative capacity, and ability to be expanded for over 68 doublings without induction of karyotypic abnormalities. Interestingly both groups found expression of the pluripotency gene OCT-4 but not NANOG.

For the current invention, a traumatic injury to the CNS is characterized by a physical impact to the central nervous system. For example, a traumatic brain injury results when the brain is subjected to a physical force that results in progressive neuronal cell damage and/or cell death and/or inflammation. A traumatic brain injury may result from a blow to the head and manifest as either an open or closed injury, these occur in civilian life or in combat situations. Severe brain damage can occur from lacerations, skull fractures, and conversely, in the absence of external signs of head injury. Accordingly, the methods of the invention can be used to treat a traumatic brain injury, including, blunt traumas, as well as, penetrating traumas such as bullet wounds. The physical forces resulting in a traumatic brain injury may cause their effects by inducing three types of injury: skull fracture, parenchymal injury, and vascular injury. Parenchymal injuries include concussion, direct parenchymal injury and diffuse axonal injury. Concussions are characterized as a clinical syndrome of alteration of consciousness secondary to head injury typically resulting from a change in the momentum of the head (movement of the head arrested against a ridged surface). The pathogenesis of sudden disruption of nervous activity is unknown, but the biochemical and physiological abnormalities that occur include, for example, depolarization due to excitatory amino acid-mediated ionic fluxes across cell membranes, depletion of mitochondrial adenosine triphosphate, and alteration in vascular permeability. Postconcussive syndrome may show evidence of direct parenchymal injury, but in some cases there is no evidence of damage.

Contusion and lacerations are conditions in which direct parenchymal injury of the brain has occurred, either through transmission of kinetic energy to the brain and bruising analogous to what is seen in soft tissue (contusion) or by penetration of an object and tearing of tissue (laceration). A blow to the surface of the brain leads to rapid tissue displacement, disruption of vascular channels, and subsequent hemorrhage, tissue injury and edema. Morphological evidence of injury in the neuronal cell body includes pyknosis of nucleus, eosinophilia of the cytoplasm, apoptosis and disintegration of the cell. Furthermore, axonal swelling can develop in the vicinity of damage neurons and also at great distances away from the site of impact.

In one embodiment of the invention, ERC are used to secrete trophic factors into the CNS of the patient. The factor is selected from the group consisting of a growth factor, a trophic factor and a cytokine. In a further embodiment, the secreted factor is selected from the group consisting of leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-.gamma., insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-.beta.), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGF.beta.-1) and TGF.beta.-3.

Recent publications indicated that mesenchymal stem cells (MSCs) transplantation is a promising therapeutical approach. MSCs transplantation can decrease inflammation and reduce production of reactive species (ROS) and matrix metalloproteinase (MMP), factors know to increase blood-brain-barrier (BBB) permeability after ICH. Preservation of BBB ameliorates the development of brain edema, a most life-threatened even after ICH. Furthermore decrease of inflammation can promote ICH-suppressed neurogenesis indirectly. MSCs secreted factors can also stimulate neurogenesis directly. MSCs have stimulating effect on angiogenesis as well. Angiogenesis and revascularization of the damaged area can support neurogenesis and migration of neurons into the lesion. Additionally MSCs have a potential to difference into neurons and may serve as an instrument for replacement therapy. These properties may contribute to brain recovery after ICH.

### EXAMPLES

### Example I

### Generation of ERC for Treatment of Neurological Indications

Collection of menstrual blood is performed according to a modification of our published procedure (Meng et al. Journal of Translational Medicine 2007, 5:57) under aseptic conditions. Manufacturing procedures take place in the General BioTechnology class 10,000 clean production suite. Each technician must properly gown when entering in the GMP room. Before entry into the clean lab area, the technician obtains a bunny suit in the ante room. After the hood of the bunny suit is placed on, they obtain a mouth covering and place on, making sure that all hair is fully covered under the hood and mouth covering. The technician then puts on a pair of sterile powder free gloves, and can enter the clean lab space with the thawed vial. Environmental monitoring is performed in the Class 10,000 clean room. The clean room uses Biological Safety Cabinets (BSC) which maintains a Class 5 environment. BSC are certified annually by an outside qualified vender. Settling plates are performed quarterly with acceptable criteria based on USP. Two settling plates are placed in the BSC during processing for a minimum of 30 minutes. Also as a negative control, a covered settling plate will be placed inside the BSC at the same time After the settling plate has been in the BSC, evaluate the plate for presence of bacterial colonies, Colony Forming Units (cfu), by allowing the plate to incubate for 48 hours. Levels requiring alert are more than 1 colony per plate. Incubator temperature should be 36-38° C. TSA plates are used to evaluate the wide spectrum of possible bacteria present. Prepared plates stored in their original wrapping at 2-8° C. should be warmed to room temperature prior to use. The product is validated from the time of manufacture to be stable at room temperature (25° C.) for 192 h (8 days).

Additionally the clean room is monitored for room temperature and particle counts. Acceptable room temperature is between 15 and 30 degrees Celsius. A MetOne Aerocet 531 particle counter and is used to evaluate the particles in the air of the clean room. It is used to confirm that the number of loose particles in the air is less than 10,000 0.5 micron particles per ft3. The particle counter is run on a weekly basis in the three major areas of the clean room space. It runs for 30 minutes in the gowning area, on the counter inside the clean room space and inside the hood. After each use of the clean room, the BSC is wiped down with 5.25% bleach then followed by a 70% isopropyl alcohol. Countertops inside the clean room space are wiped down with 70% isopropyl alcohol each day. Once a week all surfaces inside the clean room, including floor, are wiped down with enzymatic cleaner LpH using a dry disposable cloth. Yearly, all walls and ceiling are clean with a lint roller, and all soft walls are cleaned with 70% isopropyl alcohol. Before laboratory technicians are allowed into the clean room, a gowning competency must be passed. RODAC plates are utilized to assess the competency of the technician. The acceptable limits of CFU/plate are listed in the table below. This is again repeated quarterly for all qualified technicians.

Menstrual Blood Mononuclear Cell Isolation begins with the delivery of the sample to the processing lab. Washing Tube containing the menstrual blood sample is topped up to 50 ml with PBS in the Biological Safety Cabinet and cells are washed by centrifugation at 500 g for 10 minutes at room temperature, which produced a cell pellet at the bottom of the conical tube. Under sterile conditions supernatant is decanted and the cell pellet is gently dissociated by tapping until the pellet appeared liquid. The pellet is resuspended in 50 ml of PBS and gently mixed so as to produce a uniform mixture of cells in PBS. The cells are washed again by centrifugation at 500 g for 10 minutes at room temperature. Under sterile conditions, the supernatant is decanted and the cell pellet is resuspended in 15 mL complete DME/F-12 media (Hyclone) supplemented with 10% Fetal Bovine Serum (Atlas Biologicals specified to have Endotoxin level: <=100 EU/ml (levels routinely <=10 EU/ml) and hemoglobin level: <=30 mg/dl (levels routinely <=25 mg/dl). The serum lot used is sequestered and one lot is used for all experiments. Additionally, the media is supplemented with 1% penicillin/streptomycin and 0.1% amphotericin B. The sample is then plated in a T75 flask and placed in a 37° C. incubator. Media is changed after 24 hours, and then every 2-3 days at the discretion of the laboratory staff.

Once cells reach 70-80% confluence they are passaged for expansion after which they are frozen down for quarantine (minimum 2 vials) and a culture screen will be completed. The expended media from the culture will be sent for sterility and mycoplasma testing.

Screening and collection occurs at University of Indiana and is performed under approval of the local IRB. Donors are screened according to federal regulation 21 CFR1271 regarding allogeneic cell product. Specifically, healthy, non-smoking, female volunteers between 18-30 years of age sign informed consent form for providing menstrual blood sample. The volunteers undergo a standard medical history and examination including evaluation for malignancy, diabetes, leukemia, heart disease. Hematology, biochemistry, and physical examination require no abnormalities. Patients are required to be negative for anti-HIV-1, HIV-2, hepatitis B surface antigen, hepatitis B core antibody, Treponema pallidum (syphilis), CJD, antibody to trypanosome cruzi, anti-HTLV-II, Gonorrhea and Chlamydia. A collection of raw laboratory data will remain at the site and a donor case report forms are available for inspection.

### Inclusion Criteria

Menstruating women between the ages of 18 and 30

### Exclusion Criteria

### History of Toxic Shock Syndrome

Current tobacco use

### Diabetes

Positive Communicable Disease Screen (Hepatitis B or C, syphilis, chlamydia, HIV, chlamydia, gonorrhea)
Alcohol or drug abuse
Unable to disclose health history of blood-related relatives

ERC generated according to the example are characterized as menstrual blood derived, expanded cells characterized by plastic adherent cells possessing a mesenchymal-like morphology and expressing >90% the markers CD90 and CD105, while lacking expression of CD14, CD34, and CD45 (expression <5%).

### Example II

### Treatment of Intracerebral Hemorrhage Model by Intravenous Administration of ERC

As a model of traumatic brain injury, intracerebral hemorrhage was induced using the “collagenase” model originally described by Rosenberg et al, which was modified as described [22-24]. This “Collagenase” model of ICH involves using CD-1 mice. Despite of several disadvantages of the model (collagenase can cause apoptosis not typically seen in ICH; rather than producing one solid blood clot, injection of collagenase destroys small blood vessels around the injection point producing diffuse hemorrhage), collagenase model is the only model to produce spontaneous re-bleeding, which happens during the first 24 hours in many patient [25]. Additionally “collagenase” model produces more severe damage compared with others used model of ICH resulting in long-term lasting neurological deficits [10]. Therefore it is the only suitable model for long-term studies. Animals were housed with a 12-h light/dark cycle, 24° C. constant temperature, and provided unlimited access to water and rat chow. Aseptic technique was used surgery. Mice were anesthetized with ketamine (100 mg/kg, i.p.) and xylazine (10 mg/kg, i.p.) and positioned prone in a stereotaxic head frame. Temperature was maintained at 37±0.5° C. The calvarium was exposed by a midline scalp incision from the nasion to the superior nuchal line, and then the skin retracted laterally. A 1.0-mm burr hole was made 0.9 mm posterior to bregma and 1.45 mm to the right of the midline. A 26-G needle on a Hamilton syringe was inserted with stereotaxic guidance 4.0 mm into the right deep cortex/basal ganglia at a rate 1 mm/min. The collagenase (0.075 U in 0.5 μL saline) in the syringe was infused into the brain at a rate of 0.25 μL/min over 2 min with an infusion pump. The needle was left in place for an additional 10 min after injection to prevent the possible leakage of collagenase solution. After removal of the needle, the incision was closed and the mice were allowed to recover. Sham operation was performed with needle insertion only.

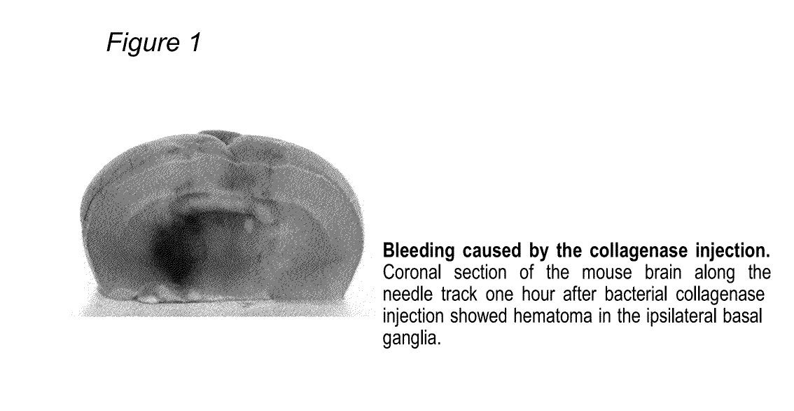

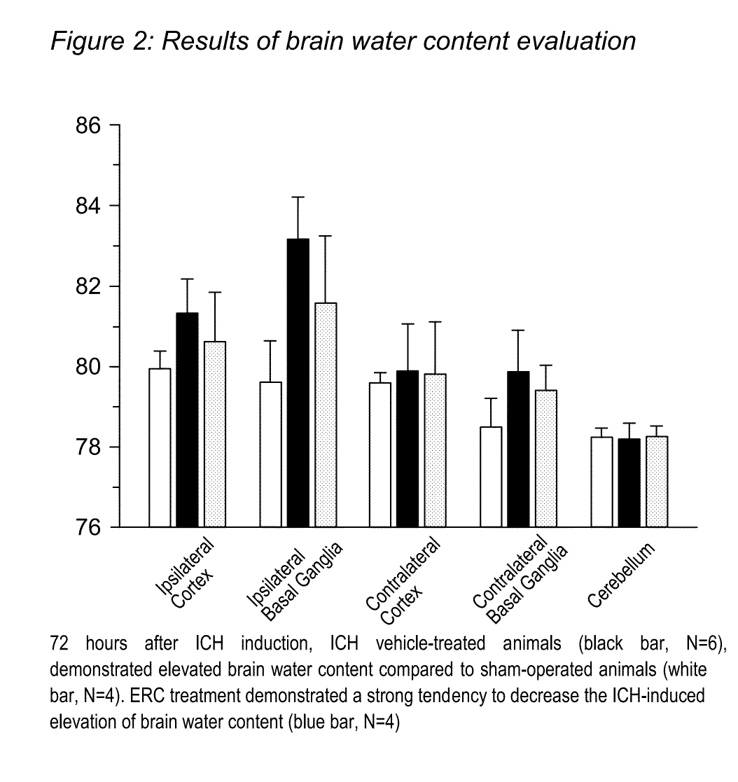

We demonstrated that shortly after collagenase injection, massive bleeding occurred (FIG. 1). Using this model system, we euthanized animals 72 hours after injection of collagenase, and brain water contents were evaluated. Development of brain edema was estimated by evaluation of brain water content via “dry-wet method”. Briefly whole brain specimens were removed immediately after euthanization of animals and divided into five parts for evaluation of brain edema: ipsilateral and contralateral basal ganglia, ipsilateral and contralateral cerebral cortex, and cerebellum. The cerebellum was used as an internal control. Tissue samples where weighed on a high-precision balance to obtain the wet weight. The tissue was then dried at 100 C for 48 h to determine the dry weight. Brain water content (%) was calculated as [(wet weight−dry weight)/wet weight]/100. A reduction in brain water in the ipsilateral basal ganglia of vehicle-treated compared to sham operated animals (79.62+/−0.51 vs 83.15+/−0.43 respectively (FIG. 2). The intravenous injection of ERC (1*106/animal) had a tendency to decrease brain water content in ERC-treated compared to vehicle-treated animals (81.65+/−0.84% vs. 83.15+/−0.43% respectively).

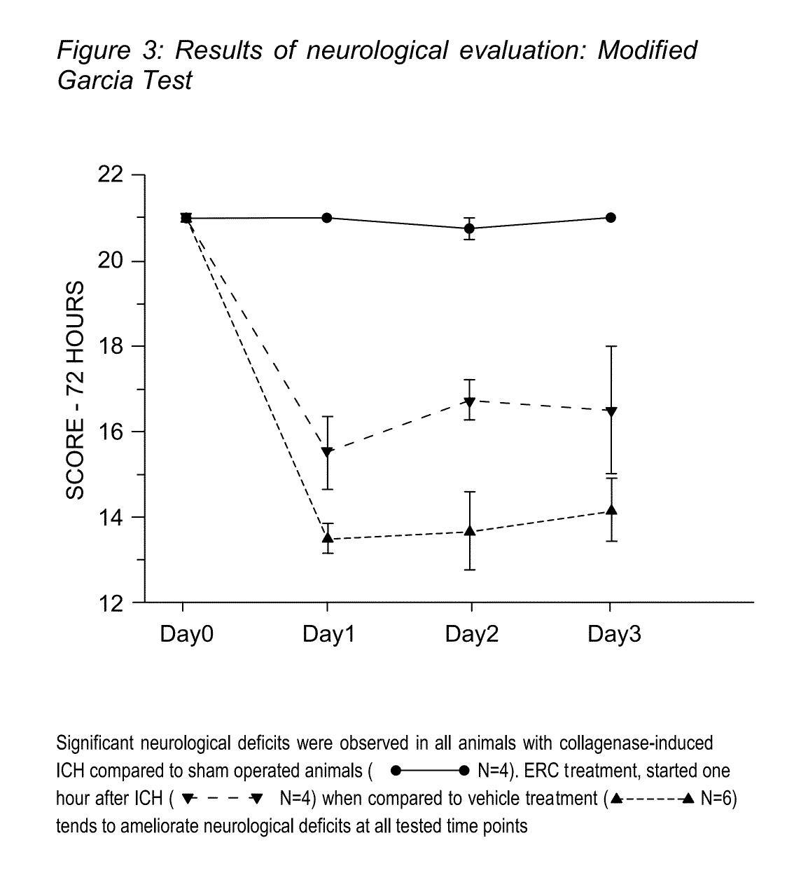

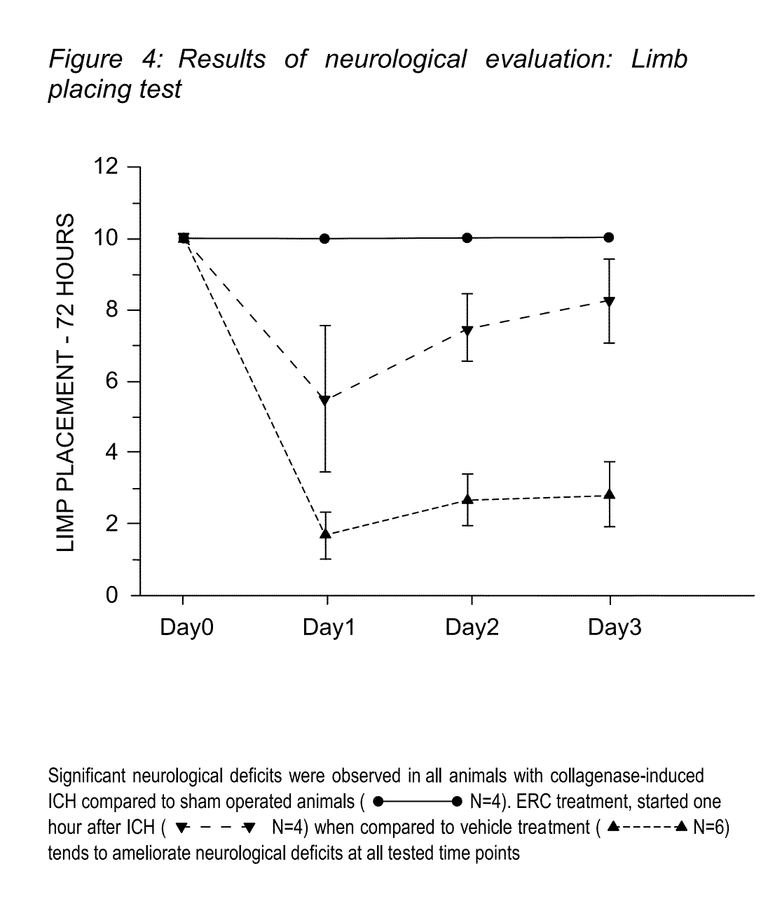

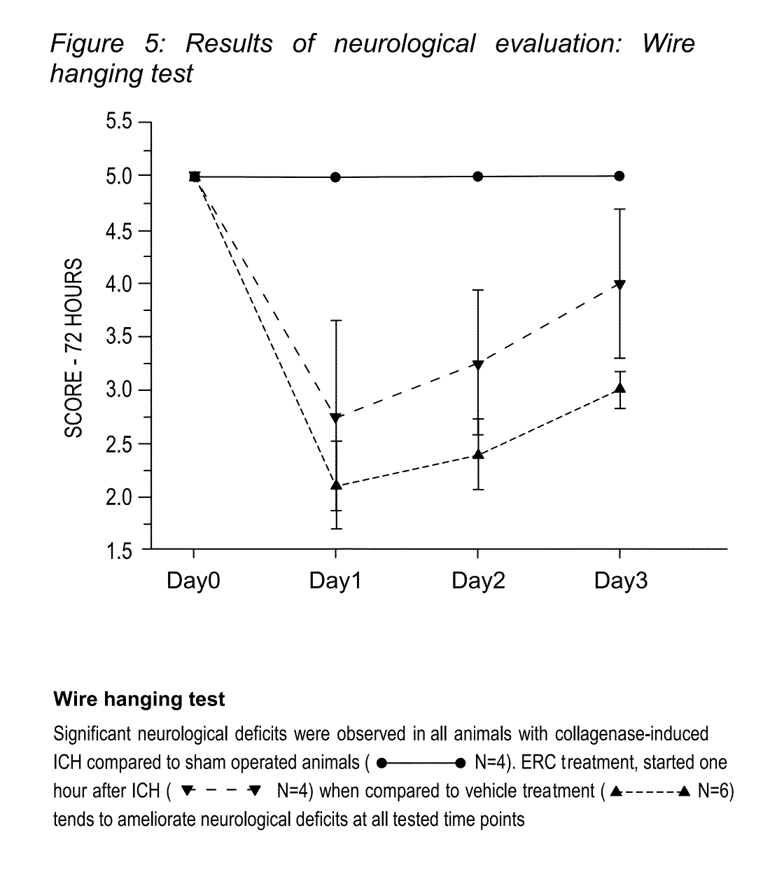

Details of the neurological tests that were performed were described previously [8, 9, 22, 23, 26, 27]. When animals were evaluated neurologically using the Garcia test, significant neurological deficits were observed in all animals with collagenase-induced ICH compared to sham operated animals (N=4). ERC treatment, started one hour after ICH (N=4) when compared to vehicle treatment (N=6) tends to ameliorate neurological deficits at all tested time points (FIG. 3). Similar improvements were noted after ERC administration using the limb placing test (FIG. 4) and the wire hanging test (FIG. 5).

It is recognized that various modifications are possible within the scope of the invention disclosed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the disclosure.

### REFERENCES

1. Arumugam, T. V., et al., Toll-like receptors in ischemia-reperfusion injury. Shock, 2009. 32(1): p. 4-16.
2. Feigin, V. L., et al., Worldwide stroke incidence and early case fatality reported in 56 population-based studies: a systematic review. Lancet Neurol, 2009. 8(4): p. 355-69.
3. Morrison, A. L., et al., Acceleration-deceleration injuries to the brain in blunt force trauma. The American journal of forensic medicine and pathology, 1998. 19(2): p. 109-12.
4. Broderick, J. P., et al., Guidelines for the management of spontaneous intracerebral hemorrhage: A statement for healthcare professionals from a special writing group of the Stroke Council, American Heart Association. Stroke, 1999. 30(4): p. 905-15.
5. Wang, J. and S. E. Tsirka, Tuftsin fragment 1-3 is beneficial when delivered after the induction of intracerebral hemorrhage. Stroke, 2005. 36(3): p. 613-8.
6. Hijioka, M., et al., Therapeutic effect of nicotine in a mouse model of intracerebral hemorrhage. J Pharmacol Exp Ther, 2011.
7. Gu, Y., C. M. Dee, and J. Shen, Interaction of free radicals, matrix metalloproteinases and caveolin-1 impacts blood-brain barrier permeability. Front Biosci (Schol Ed), 2011. 3: p. 1216-31.
8. Ma, Q., et al., Vascular adhesion protein-1 inhibition provides antiinflammatory protection after an intracerebral hemorrhagic stroke in mice. J Cereb Blood Flow Metab, 2011. 31(3): p. 881-93.
9. Lekic, T., et al., Protective effect of melatonin upon neuropathology, striatal function, and memory ability after intracerebral hemorrhage in rats. J Neurotrauma, 2010. 27(3): p. 627-37.
10. MacLellan, C. L., et al., Intracerebral hemorrhage models in rat: comparing collagenase to blood infusion. J Cereb Blood Flow Metab, 2008. 28(3): p. 516-25.
11. Masuda, T., et al., Increase in neurogenesis and neuroblast migration after a small intracerebral hemorrhage in rats. Neurosci Lett, 2007. 425(2): p. 114-9.
12. Hua, Y., et al., Thrombin and brain recovery after intracerebral hemorrhage. Stroke, 2009. 40(3 Suppl): p. S88-9.
13. Yang, S., et al., Effects of thrombin on neurogenesis after intracerebral hemorrhage. Stroke, 2008. 39(7): p. 2079-84.
14. Barreto, G., et al., Astrocytes: targets for neuroprotection in stroke. Central nervous system agents in medicinal chemistry, 2011. 11(2): p. 164-73.
15. Meng, X., et al., Endometrial regenerative cells: a novel stem cell population. Journal of translational medicine, 2007. 5: p. 57.
16. Nikoo, S., et al., Effect of menstrual blood-derived stromal stem cells on proliferative capacity of peripheral blood mononuclear cells in allogeneic mixed lymphocyte reaction. The journal of obstetrics and gynaecology research, 2012.
17. Zemel'ko, V. I., et al., [Multipotent mesenchymal stem cells of desquamated endometrium: isolation, characterization and use as feeder layer for maintenance of human embryonic stem cell lines]. Tsitologiia, 2011. 53(12): p. 919-29.
18. Kazemnejad, S., et al., Characterization and chondrogenic differentiation of menstrual blood-derived stem cells on a nanofibrous scaffold. The International journal of artificial organs, 2012. 35(1): p. 55-66.
19. Rodrigues, M. C., et al., Toward personalized cell therapies: autologous menstrual blood cells for stroke. Journal of biomedicine & biotechnology, 2011. 2011: p. 194720.
20. Cui, C. H., et al., Menstrual blood-derived cells confer human dystrophin expression in the murine model of Duchenne muscular dystrophy via cell fusion and myogenic transdifferentiation. Molecular biology of the cell, 2007. 18(5): p. 1586-94.
21. Patel, A. N., et al., Multipotent menstrual blood stromal stem cells: isolation, characterization, and differentiation. Cell transplantation, 2008. 17(3): p. 303-11.
22. Manaenko, A., et al., Arginine-vasopressin V1a receptor inhibition improves neurologic outcomes following an intracerebral hemorrhagic brain injury. Neurochem Int, 2011. 58(4): p. 542-8.
23. Manaenko, A., et al., Effect of gap junction inhibition on intracerebral hemorrhage-induced brain injury in mice. Neurol Res, 2009. 31(2): p. 173-8.
24. Manaenko, A., et al., Geldanamycin reduced brain injury in mouse model of intracerebral hemorrhage. Acta Neurochir Suppl, 2011. 111: p. 161-5.
25. Fujii, Y., et al., Hematoma enlargement in spontaneous intracerebral hemorrhage. J Neurosurg, 1994. 80(1): p. 51-7.
26. Manaenko, A., et al., Comparison Evans Blue injection routes: Intravenous versus intraperitoneal, for measurement of blood-brain barrier in a mice hemorrhage model. J Neurosci Methods, 2011. 195(2): p. 206-10.
27. Manaenko, A., et al., Heat shock protein 70 upregulation by geldanamycin reduces brain injury in a mouse model of intracerebral hemorrhage. Neurochem Int, 2010. 57(7): p. 844-50.

What is claimed is:

1. A method of treating a neurological injury comprising administering into a patient an effective amount of endometrial regenerative cells.

2. The method of claim 1, wherein said neurological injury is acute or chronic.

3. The method of claim 2, wherein said acute neurological injury is selected from the group consisting of: a) traumatic brain injury; b) stroke; and c) intracerebral hemorrhage.

4. The method of claim 1, wherein said endometrial regenerative cells originate from the endometrium and said cell expresses a marker selected from the group consisting of: CD29, CD41a, CD44, CD90, and CD105, and have an ability to proliferate at a rate of 0.5-1.5 doublings per 24 hours in a growth medium.

5. The method of claim 1, wherein said cell further expresses a marker selected from the group consisting of: NeuN, CD9, CD62, CD59, Actin, GFAP, NSE, Nestin, CD73, SSEA-4, hTERT, Oct-4, and tubulin.

6. The method of claim 1, wherein said cell further expresses a marker selected from hTERT and Oct-4, but does not express a STRO-1 marker, and has an ability to undergo cell division in less than 24 hours in a growth medium.

7. The method of claim 1, wherein said cell further expresses a STRO-1 marker, and has an ability to proliferate at a rate of 0.5-0.9 doublings per 24 hours in a growth medium.

8. The method of claim 1, wherein said cell produces a compound selected from the group consisting of: matrix metalloprotease 3 (MMP3), matrix metalloprotease 10 (MMP10), GM-CSF, PDGF-BB, and angiogenic factor ANG-2.

9. The method of claim 1, wherein said endometrial regenerative cell expresses substantially higher amounts of the following genes as compared to bone marrow derived mesenchymal stem cells: somatostatin receptor 1, forkhead box L2, FAM105A, synaptopodin 2-like, anoctamin 4, spondin 2, CARD16, VAT1L, indolethylamine N-methyltransferase, deiodinase, iodothyronine, type II (DIO2), aldehyde dehydrogenase 1 family, member A1, 5-hydroxytryptamine (serotonin) receptor 2B, caspase recruitment domain family, member 17, Rho GTPase activating protein 20, zinc finger and BTB domain containing 46 (ZBTB46), synaptopodin 2-like (SYNPO2L), transcript variant 1, mRNA, chromosome 13 open reading frame 15 (C13orf15), mRNA, homeobox D11 (HOXD11), mRNA, oxidized low density lipoprotein (lectin-like) receptor 1 (OLR1), transcript variant 2, mRNA, homeobox D10 (HOXD10), mRNA, Pregnancy specific beta-1-glycoprotein 4, matrix metallopeptidase 3 (stromelysin 1, progelatinase) (MMP3), mRNA, actin filament associated protein 1-like 2 (AFAP1L2), transcript variant 2, mRNA,SH3-domain GRB2-like (endophilin) interacting protein 1 (SGIP1), mRNA, solute carrier family 7 (cationic amino acid transporter, y+ system), member 2 (SLC7A2), transcript variant 1, mRNA, pregnancy specific beta-1-glycoprotein 8 (PSG8), transcript variant 3, mRNA, Wilms tumor 1 (WT1), transcript variant F, mRNA, platelet-derived growth factor beta polypeptide (PDGFB), transcript variant 1, mRNA, forkhead box F1 (FOXF1), mRNA, mannan-binding lectin serine peptidase 1 (C4/C2 activating component of Ra-reactive factor) (MASP1), transcript variant 1, mRNA, deiodinase, iodothyronine, type II (DIO2), transcript variant 4, mRNA, G protein-coupled receptor 126 (GPR126), transcript variant b1, mRNA, stimulated by retinoic acid gene 6 homolog (mouse) (STRA6), transcript variant 6, mRNA, hydroxysteroid (11-beta) dehydrogenase 1 (HSD11B1), transcript variant 2, mRNA, RAS guanyl releasing protein 2 (calcium and DAG-regulated) (RASGRP2), transcript variant 4, mRNA, membrane bound O-acyltransferase domain containing 1 (MBOAT1), mRNA, LON peptidase N-terminal domain and ring finger 2 (LONRF2), mRNA, interferon-induced protein with tetratricopeptide repeats 2 (IFIT2), mRNA, cathepsin C (CTSC), transcript variant 3, mRNA, caspase 1, apoptosis-related cysteine peptidase (interleukin 1, beta, convertase) (CASP1), transcript variant beta, mRNA, integrin, alpha 4 (antigen CD49D, alpha 4 subunit of VLA-4 receptor) (ITGA4), mRNA, integrin, alpha 6 (ITGA6), transcript variant 1, mRNA, contactin 3 (plasmacytoma associated) (CNTN3), mRNA, synaptophysin-like 2 (SYPL2), mRNA, Rho GTPase activating protein 25 (ARHGAP25), transcript variant 4, mRNA, potassium voltage-gated channel, Shal-related subfamily, member 2 (KCND2), mRNA, S100 calcium binding protein A4 (S100A4), transcript variant 1, mRNA, chemokine (C-C motif) receptor-like 2 (CCRL2), transcript variant 2, mRNA, integrin, alpha 6 (ITGA6), transcript variant 2, mRNA, guanylate binding protein 4 (GBP4), mRNA, multiple C2 domains, transmembrane 1 (MCTP1), transcript variant S, mRNA, interferon-induced protein with tetratricopeptide repeats 2 (IFIT2), mRNA, Clq and tumor necrosis factor related protein 9 (C1QTNF9), mRNA, progesterone receptor (PGR), transcript variant 2, mRNA, hypothetical LOC646113 (FLJ43390), non-coding RNA. zinc finger and BTB domain containing 46 (ZBTB46), mRNA, interferon-induced protein with tetratricopeptide repeats 3 (IFIT3), transcript variant 1, mRNA, microphthalmia-associated transcription factor (MITF), transcript variant 2, mRNA, interleukin 24 (IL24), transcript variant 4, mRNA, neuronal pentraxin I (NPTX1), mRNA, integrin, alpha 4 (antigen CD49D, alpha 4 subunit of VLA-4 receptor) (ITGA4), mRNA, WT1 antisense RNA (non-protein coding) (WT1-AS), non-coding RNA, microphthalmia-associated transcription factor (MITF), transcript variant 2, mRNA, sterile alpha motif domain containing 12 (SAMD12), transcript variant 1, mRNA, chromosome 7 open reading frame 58 (C7orf58), transcript variant 1, mRNA, phospholipase B domain containing 1 (PLBD1), mRNA, podocalyxin-like (PODXL), transcript variant 2, mRNA, interleukin 8 (IL8), mRNA, chromosome 7 open reading frame 58 (C7orf58), transcript variant 2, mRNA, protein kinase, cAMP-dependent, regulatory, type II, beta (PRKAR2B), mRNA, chromosome 8 open reading frame 4 (C8orf4), mRNA, stimulated by retinoic acid gene 6 homolog (mouse) (STRA6), transcript variant 5, mRNA, progesterone receptor (PGR), transcript variant 2, mRNA, angiopoietin 2 (ANGPT2), transcript variant 2, mRNA, synaptophysin-like 2 (SYPL2), mRNA, retinoic acid receptor responder (tazarotene induced) 2 (RARRES2), mRNA, claudin 1 (CLDN1), mRNA, sal-like 1 (Drosophila) (SALL1), transcript variant 1, mRNA, calcium channel, voltage-dependent, T type, alpha 1H subunit (CACNA1H), transcript variant 2, mRNA, armadillo repeat containing 4 (ARMC4), mRNA, phosphatidylinositol-specific phospholipase C, X domain containing 3 (PLCXD3), mRNA, G protein-coupled receptor, family C, group 5, member A (GPRC5A), mRNA, GATA binding protein 2 (GATA2), transcript variant 1, mRNA, actin filament associated protein 1-like 1 (AFAP1L1), transcript variant 1, mRNA, ST6 (alpha-N-acetyl-neuraminyl-2,3-beta-galactosyl-1, 3)-N-acetylgalactosaminide alpha-2,6-sialyltransferase 5 (ST6GALNAC5), mRNA, H19, imprinted maternally expressed transcript (non-protein coding) (H19), non-coding RNA, sphingosine-l-phosphate receptor 3 (S1PR3), mRNA, protein kinase, cAMP-dependent, regulatory, type II, beta (PRKAR2B), mRNA, interleukin 7 receptor (IL7R), mRNA, calcium/calmodulin-dependent protein kinase IG (CAMK1G), mRNA, homeobox A11 (HOXA11), mRNA, renin (REN), mRNA, transcription factor AP-2 gamma (activating enhancer binding protein 2 gamma) (TFAP2C), mRNA, cathepsin C (CTSC), transcript variant 2, mRNA, STAM binding protein-like 1 (STAMBPL1), mRNA, Hypothetical protein LOC254057, adrenomedullin (ADM), mRNA, tenascin XB (TNXB), transcript variant XB-S, mRNA, KH domain containing, RNA binding, signal transduction associated 3 (KHDRBS3), mRNA, solute carrier family 35, member F3 (SLC35F3), mRNA, collagen, type IV, alpha 6 (COL4A6), transcript variant B, mRNA, protocadherin 7 (PCDH7), transcript variant a, mRNA, chromosome 10 open reading frame 58 (C10orf58), transcript variant 1, mRNA, fibroblast growth factor 9 (glia-activating factor) (FGF9), mRNA, sushi domain containing 3 (SUSD3), mRNA, melanoma associated antigen (mutated) 1-like 1 (MUM1L1), transcript variant 2, mRNA, keratin 18 pseudogene (F1140504), non-coding RNA, C-type lectin domain family 14, member A (CLEC14A), mRNA, 1-acylglycerol-3-phosphate O-acyltransferase 9 (AGPAT9), mRNA, phosphodiesterase 8B (PDE8B), transcript variant 3, mRNA, ArfGAP with dual PH domains 2 (ADAP2), mRNA, Keratin 18, family with sequence similarity 65, member C (FAM65C), mRNA, androgen receptor (AR), transcript variant 1, mRNA, phosphodiesterase 9A (PDE9A), transcript variant 2, mRNA, Intercellular adhesion molecule 1, monoglyceride lipase (MGLL), transcript variant 2, mRNA, HOXA11 antisense RNA 1 (non-protein coding) (HOXA11-AS1), antisense RNA, receptor (chemosensory) transporter protein 4 (RTP4), mRNA, reticulon 4 receptor (RTN4R), mRNA, Keratin pseudogene, annexin A3 (ANXA3), mRNA, RAS guanyl releasing protein 2 (calcium and DAG-regulated) (RASGRP2), transcript variant 3, mRNA, serum deprivation response (SDPR), mRNA, collagen, type XIII, alpha 1 (COL13A1), transcript variant 21, mRNA, v-ets erythroblastosis virus E26 oncogene homolog 2 (avian) (ETS2), mRNA, ADAM metallopeptidase domain 8 (ADAMS), transcript variant 3, mRNA, prostate collagen triple helix (PCOTH), transcript variant 1, mRNA, family with sequence similarity 124A (FAM124A), transcript variant 1, mRNA, interferon, alpha-inducible protein 27 (IF127), transcript variant 2, mRNA, hypothetical protein MGC16121 (MGC16121), non-coding RNA, egf-like module containing, mucin-like, hormone receptor-like 2 (EMR2), transcript variant 4, mRNA, RAS guanyl releasing protein 2 (calcium and DAG-regulated) (RASGRP2), transcript variant 3, mRNA, KH domain containing, RNA binding, signal transduction associated 3 (KHDRBS3), mRNA, chromosome 9 open reading frame 47 (C9orf47), transcript variant 1, mRNA, family with sequence similarity 162, member B (FAM162B), mRNA, sodium channel, voltage-gated, type IX, alpha subunit (SCN9A), mRNA, glutaredoxin (thioltransferase) (GLRX), transcript variant 2, mRNA, four jointed box 1 (Drosophila) (FJX1), mRNA, chromosome 10 open reading frame 58 (C10orf58), transcript variant 1, mRNA, archaelysin family metallopeptidase 1 (AMZ1), mRNA, laminin, alpha 5 (LAMAS), mRNA, myosin XVI (MYO16), transcript variant 2, mRNA, SH3-domain GRB2-like (endophilin) interacting protein 1 (SGIP1), mRNA, nuclear receptor subfamily 2, group F, member 1 (NR2F1), mRNA, GULP, engulfment adaptor PTB domain containing 1 (GULP1), mRNA, actin binding LIM protein family, member 3 (ABLIM3), mRNA, transmembrane protein 154 (TMEM154), mRNA, germ cell associated 1 (GSG1), transcript variant 1, mRNA, suppressor of cytokine signaling 2 (SOCS2), mRNA, GULP, engulfment adaptor PTB domain containing 1 (GULP1), mRNA, integrin, alpha 4 (antigen CD49D, alpha 4 subunit of VLA-4 receptor) (ITGA4), mRNA, DENN/MADD domain containing 2A (DENND2A), mRNA, interferon-induced protein with tetratricopeptide repeats 1 (IFIT1), transcript variant 2, mRNA, C-type lectin domain family 2, member B (CLEC2B), mRNA, hairy and enhancer of split 5 (Drosophila) (HESS), mRNA, chromosome 7 open reading frame 58 (C7orf58), transcript variant 2, mRNA, Keratin 18, cathepsin C (CTSC), transcript variant 1, mRNA, G protein-coupled receptor 183 (GPR183), mRNA, Mitogen-activated protein kinase kinase kinase 8 caspase recruitment domain family, member 9 (CARD9), transcript variant 2, mRNA, nidogen 1 (NID1), mRNA, adenosine monophosphate deaminase 3 (AMPD3), transcript variant 3, mRNA, opioid growth factor receptor-like 1 (OGFRL1), mRNA, interferon induced transmembrane protein 1 (9-27) (IFITM1), mRNA, interleukin 2 receptor, beta (IL2RB), mRNA, tripartite motif containing 14 (TRIM14), transcript variant 1, mRNA, acyl-CoA synthetase short-chain family member 1 (ACSS1), nuclear gene encoding mitochondrial protein, mRNA, RNA binding motif protein 24 (RBM24), transcript variant 3, mRNA, stathmin-like 2 (STMN2), transcript variant 1, mRNA, GULP, engulfment adaptor PTB domain containing 1 (GULP1), mRNA, secreted and transmembrane 1 (SECTM1), mRNA, betaine-homocysteine S-methyltransferase 2 (BHMT2), transcript variant 1, mRNA, lymphocyte-activation gene 3 (LAG3), mRNA, transmembrane protein 51 (TMEM51), transcript variant 1, mRNA, guanine nucleotide binding protein (G protein), gamma 11 (GNG11), mRNA, CD163 molecule-like 1 (CD163L1), mRNA, major histocompatibility complex, class I, F (HLA-F), transcript variant 2, mRNA, endoplasmic reticulum metallopeptidase 1 (ERMP1), mRNA, cytochrome b5 reductase 2 (CYB5R2), mRNA, mitogen-activated protein kinase kinase kinase 5 (MAP3K5), mRNA, interleukin 20 (IL20), mRNA, T-box 3 (TBX3), transcript variant 1, mRNA, adrenergic, alpha-1D-, receptor (ADRA1D), mRNA, leucine rich repeat containing 8 family, member C (LRRC8C), mRNA, folate receptor 3 (gamma) (FOLR3), mRNA, tumor necrosis factor receptor superfamily, member 21 (TNFRSF21), mRNA, actin binding LIM protein family, member 3 (ABLIM3), mRNA, CD44 molecule (Indian blood group) (CD44), transcript variant 7, mRNA, phosphatidylinositol-3,4,5-trisphosphate-dependent Rac exchange factor 1 (PREX1), mRNA, tripartite motif containing 14 (TRIM14), transcript variant 1, mRNA, aldehyde dehydrogenase 1 family, member A1 (ALDH1A1), mRNA, adaptor-related protein complex 1, mu 2 subunit (AP1M2), mRNA, integrin, alpha 1 (ITGA1), mRNA, mitogen-activated protein kinase kinase kinase 5 (MAP3K5), mRNA, serpin peptidase inhibitor, clade B (ovalbumin), member 2 (SERPINB2), transcript variant 2, mRNA, scavenger receptor class B, member 1 (SCARB1), transcript variant 1, mRNA, homeobox D9 (HOXD9), mRNA, G protein-coupled receptor, family C, group 5, member B (GPRC5B), mRNA, Rho guanine nucleotide exchange factor (GEF) 16 (ARHGEF16), mRNA, adrenergic, alpha-2A-, receptor (ADRA2A), mRNA, intercellular adhesion molecule 4 (Landsteiner-Wiener blood group) (ICAM4), transcript variant 2, mRNA, guanine nucleotide binding protein (G protein), gamma 4 (GNG4), transcript variant 1, mRNA, BCL2-like 10 (apoptosis facilitator) (BCL2L10), mRNA, monoglyceride lipase (MGLL), transcript variant 1, mRNA, Furry homolog (Drosophila) phorbol-12-myristate-13-acetate-induced protein 1 (PMAIP1), mRNA, transmembrane and tetratricopeptide repeat containing 1 (TMTC1), transcript variant 1, mRNA, collagen, type IV, alpha 5 (COL4A5), transcript variant 1, mRNA, disrupted in renal carcinoma 3 (DIRC3), non-coding RNA, Rho GDP dissociation inhibitor (GDI) beta (ARHGDIB), mRNA, oxytocin receptor (OXTR), mRNA, tumor necrosis factor receptor superfamily, member 6b, decoy (TNFRSF6B), mRNA, cytochrome b5 reductase 2 (CYB5R2), mRNA, peptidase M20 domain containing 2 (PM20D2), mRNA, shroom family member 3 (SHROOM3), mRNA, family with sequence similarity 46, member C (FAM46C), mRNA, nuclear factor of kappa light polypeptide gene enhancer in B-cells inhibitor, epsilon (NFKBIE), mRNA, tumor necrosis factor receptor superfamily, member 6b, decoy (TNFRSF6B), mRNA.
